(19) United States
(12) Patent Application Publication
(10) Pub. No.: US 2022/0361852 A1
(43) Pub. Date: Nov. 17, 2022

(54) ULTRASONIC DIAGNOSTIC APPARATUS AND DIAGNOSIS ASSISTING METHOD

(71) Applicant: FUJIFILM Healthcare Corporation, Kashiwa-shi (JP)

(72) Inventor: Yoko Fujihara, Chiba (JP)

(21) Appl. No.: 17/726,628

(22) Filed: Apr. 22, 2022

(30) Foreign Application Priority Data
May 12, 2021 (JP) 2021-080736

(51) Int. Cl. A61B 8/08; G06T 7/00

(52) U.S. Cl. CPC A61B 8/5292; A61B 8/085; G06T 7/0012; G06T 2207/30096; G06T 2207/10132

(57) ABSTRACT

An image analysis unit has a reliability calculation unit that calculates the reliability indicating the possibility that a lesion candidate is a lesion. A mark display control unit displays a mark reporting the lesion candidate on an ultrasonic image. In a continuous detection state where the reliability continues to meet the candidate detection conditions, the mark is continued to be displayed. During the time, the form of the mark is fixed from the time point when the candidate detection conditions are first met until a fixed form period elapses. The form of the mark is then changed as the reliability varies.

### CROSS REFERENCE TO RELATED APPLICATION

This application claims priority to Japanese Patent Application No. 2021-080736 filed on May 12, 2021, which is incorporated herein by reference in its entirety including the specification, claims, drawings, and abstract.

### TECHNICAL FIELD

The present disclosure relates to an ultrasonic diagnostic apparatus and a diagnosis assisting method, and in particular to a technique of notifying an examiner of a lesion candidate.

### BACKGROUND

In an ultrasonic examination, a probe is brought into contact with a surface of the subject and is scanned along the surface of the subject. In the process of scanning, a real-time tomographic image displayed on a display is observed by the examiner, and the presence or absence of a lesion is judged through the observation. When a lesion is found, it is examined in detail.

It is not easy to visually identify a lesion which momentarily appears on a dynamically changing tomographic image. Computer Aided Detection (CADe) is known as a technique for assisting identification of a lesion. This technique detects, for example, a lesion candidate included in a tomographic image and notifies the examiner of the detected lesion candidate. For example, a mark surrounding the lesion candidate is displayed on the tomographic image. CADe is used along with Computer Aided Diagnosis (CAD) or included in CAD. CAD may also be denoted as CADx.

Patent Document 1 (JP 2004-159739 A) discloses an ultrasonic diagnostic apparatus having a CAD function. Patent Document 1 nowhere discloses changing a display mode of a mark for reporting a lesion candidate. Patent Document 2 (WO 2018/198327 A) discloses that an ultrasonic diagnostic apparatus having the CAD function changes a display mode of a mark for reporting a lesion candidate. However, this technique is provided for preventing examiners from overlooking lesion candidates, on the assumption that different examiners have different tendencies to overlook the lesion candidates. That is, the technique is independent of the likelihood of a lesion candidate being a lesion. Patent Document 3 (JP 2012-249964 A) describes that an ultrasonic diagnostic apparatus having the CAD function displays a diagnosis name when the reliability of detection of a lesion candidate is equal to or higher than a threshold.

In the present description, a lesion refers to a site that may be diseased or needs to be examined in detail. A lesion candidate refers to a site detected to assist identification and diagnosis of a lesion by the examiner.

### CITATION LIST

### Patent Literature

Patent Document 1: JP 2004-159739 A
Patent Document 2: WO 2018/198327 A
Patent Document 3: JP 2012-249964 A

### SUMMARY

In displaying a mark reporting a lesion candidate, the reliability of detection of the lesion candidate may be expressed by changing the form of the mark. When this technique is adopted, the form of the mark tends to change immediately after the start of displaying the mark. This is because, in the initial stage of lesion candidate detection, a detection state tends to be unstable, and the reliability thus tends to be changeable. The change in form of the mark may interfere with observation of an ultrasonic image, and specifically, it may be obtrusive to the examiner who is observing the ultrasonic image.

An object of the present disclosure is to limit an undesired change in form of a mark reporting a lesion candidate when the reliability of detection of the lesion candidate is expressed by changing the form of the mark.

An ultrasonic diagnostic apparatus according to the present disclosure includes a calculation unit that receives an array of frame data obtained by repeating scanning of an ultrasound beam and calculates, for each set of frame data, the reliability indicating the possibility that a lesion candidate included in the frame data is a lesion, and a display control unit that displays a mark reporting the lesion candidate on an ultrasonic image formed based on the array of frame data and continues to display the mark in a continuous detection state where the lesion candidate is detected continuously. In the continuous detection state, the display control unit fixes the form of the mark from the time point when the lesion candidate is first detected until a fixed form period elapses, and changes the form of the mark according to the reliability after the fixed form period elapses.

A diagnosis assisting method according to the present disclosure includes the steps of calculating the reliability indicating the possibility that a lesion candidate included in frame data is a lesion, for each set of frame data constituting an array of frame data obtained by repeating scanning of an ultrasound beam, and displaying a mark reporting the lesion candidate on an ultrasonic image formed based on the array of frame data. In a continuous detection state where the lesion candidate is detected continuously, the mark is continued to be displayed, and while the mark is continued to be displayed, the form of the mark is fixed from the time point when the lesion candidate is first detected until a fixed form period elapses and is then changed according to the reliability.

### BRIEF DESCRIPTION OF DRAWINGS

Embodiments of the present disclosure will be described based on the following figures, wherein:

FIG. 1 is a block diagram showing an ultrasonic diagnostic apparatus according to an embodiment;

FIG. 2 is a diagram for explaining a method of generating a mark;

FIG. 3 is a flowchart showing an operation example relating to display of the mark;

FIG. 4 is a diagram showing a first example of mark display control;

FIG. 5 is a diagram for explaining the first example in more detail;

FIG. 6 is a diagram showing a second example of mark display control;

FIG. 7 is a diagram showing a variant of the ultrasonic diagnostic apparatus; and

FIG. 8 is a diagram showing a third example of mark display control.

### DESCRIPTION OF EMBODIMENTS

Hereinafter, embodiments will be described with reference to the drawings.

### (1) Summary of Embodiments

An ultrasonic diagnostic apparatus according to an embodiment has a calculation unit and a display control unit. The calculation unit receives an array of frame data obtained by repeating scanning of an ultrasound beam and calculates, for each set of frame data, the reliability indicating the possibility that a lesion candidate included in the frame data is a lesion. The display control unit displays a mark reporting the lesion candidate on an ultrasonic image formed based on the array of frame data. In a continuous detection state where the lesion candidate is detected continuously, the mark is continued to be displayed. In the continuous detection state, the display control unit fixes the form of the mark from the time point when the lesion candidate is first detected until a fixed form period elapses, and then changes the form of the mark according to the reliability after the fixed form period elapses. The calculation unit corresponds to a calculator. The display control unit corresponds to a controller.

According to the above configuration, when the lesion candidate is detected, the form of the mark is fixed until the fixed form period elapses. The reliability is usually unstable in the initial stage of lesion candidate detection, so that display of the reliability is stopped during that period. This makes it possible to avoid problems caused by a change in form of the mark in the initial stage of detection, such as, for example, a problem that the change in form of the mark is obtrusive to the examiner. The examiner is thus allowed to concentrate on observation of a site reported by the mark in the initial stage of detection. When the initial detection stage has passed; that is, when the fixed form period has elapsed, the form of the mark is usually changed slowly, if it is changed, so that the above problem is unlikely to occur. Typically, the initial stage of detection is a period in which the probe is moved relatively quickly, and after that period, the probe is stopped, and the lesion candidate is then observed in detail while the position and posture of the probe are slowly adjusted.

In the embodiment, the display control unit changes, in the continuous detection state, the form of the mark in a stepwise manner as the reliability increases after the fixed form period elapses. Although the form of the mark may be changed in a continuous manner, by changing the form of the mark in a stepwise manner, changes in reliability can be recognized more easily.

In the embodiment, a non-display range and a display range are defined on the reliability axis. The display range is divided into a plurality of sections including a lowest section. During the fixed form period, the mark takes an initial form. After the fixed form period elapses, the form of the mark is any one of a plurality of forms corresponding to the plurality of sections. The plurality of forms include the initial form which corresponds to the lowest section. Making the form of the mark displayed during the fixed form period the same as the form of the mark corresponding to the lowest section allows a reduction in the frequency of form change at the time point when the fixed form period elapses.

In the embodiment, changing the form of the mark in a stepwise manner includes at least one of changing the thickness of the mark in a stepwise manner, changing a color of the mark in a stepwise manner, changing the transparency of the mark in a stepwise manner, and changing a type of a line constituting the mark in a stepwise manner. The mark may be composed of four display elements for identifying four corners of an area containing the lesion candidate. The mark may also be composed of a painted area.

In the embodiment, the display control unit displays the mark when the reliability becomes greater than a threshold and erases the mark when the reliability becomes smaller than the threshold. The mark on the ultrasonic image is erased when the reliability becomes smaller than the threshold before the fixed form period elapses. This makes it possible to report the fact of momentary detection and, at the same time, prevent the mark from interfering with observation of the ultrasonic image.

In the embodiment, the calculation unit judges, for each set of frame data, an attribute of the lesion candidate included in the frame data. In the continuous detection state, the display control unit changes the form of the mark according to a combination of the reliability and the attribute after the fixed form period elapses. This makes it possible to recognize the magnitude of reliability and recognize the attribute of the lesion candidate through the form of the mark. The concept of attributes involves, for example, a disease name, a disease state, the grade of malignancy, and the like.

In the embodiment, the calculation unit judges, for each set of frame data, whether the attribute of the lesion candidate included in the frame data is an important attribute or an unimportant attribute. When a judgment that the attribute is an important attribute is obtained, the display control unit changes, in the continuous detection state, the form of the mark according to the reliability after the fixed form period elapses. On the other hand, when a judgment that the attribute is an unimportant attribute is obtained, the display control unit erases, in the continuous detection state, the mark after the fixed form period elapses. This makes it possible to avoid excessive reporting on a certain tissue that does not require careful examination.

A diagnosis assisting method according to an embodiment has a calculation step and a display step. In the calculation step, the reliability indicating the possibility that a lesion candidate included in frame data is a lesion is calculated for each set of frame data constituting an array of frame data obtained by repeating scanning of an ultrasound beam. In the display step, a mark reporting the lesion candidate is displayed on an ultrasonic image formed based on the array of frame data. In a continuous detection state where the lesion candidate is detected continuously, the mark is continued to be displayed. While the mark is continued to be displayed, the form of the mark is fixed from the time point when the lesion candidate is first detected until a fixed form period elapses and is then changed according to the reliability. A program according to an embodiment is a program for executing the above calculation assisting method on an information processing device.

The information processing device has a non-transitory storage medium storing the program. The information processing device is, for example, an ultrasonic diagnostic apparatus, an ultrasonic diagnostic system, a computer, or the like.

### (2) Details of Embodiments

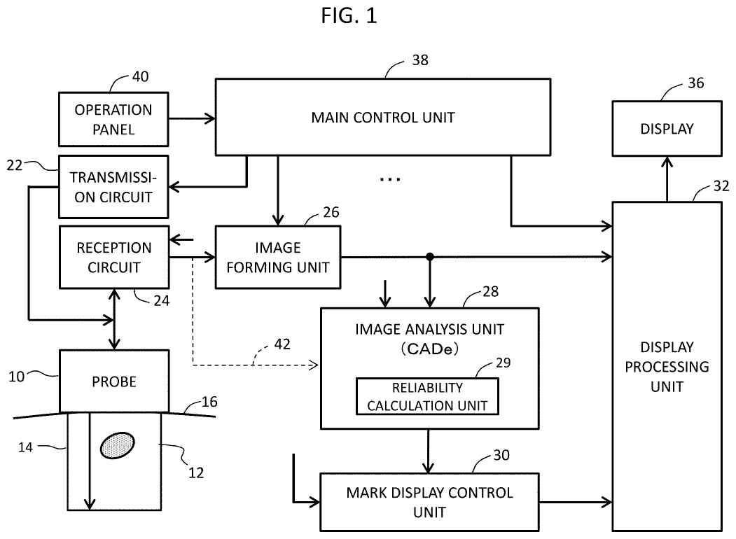

FIG. 1 shows a block diagram of a configuration of an ultrasonic diagnostic apparatus according to an embodiment. The ultrasonic diagnostic apparatus is a medical apparatus that is placed in a medical institution, such as a hospital, and generates an ultrasonic image based on received signals that are obtained by transmitting and receiving ultrasonic waves to and from a living body (subject). An organ to be diagnosed by ultrasound is a breast, for example.

In mass screening of breast diseases, it is necessary to identify a lesion in a short time without overlooking. The ultrasonic diagnostic apparatus according to the embodiment has a CADe function of automatically detecting a lesion candidate (for example, a low brightness area that may be a tumor) included in an ultrasonic image, in order to assist the examiner in identifying a lesion. This function will be described below in detail.

A probe 10 functions as means for transmitting and receiving ultrasonic waves. Specifically, the probe 10 is a portable transmitter/receiver and is held and operated by the examiner (a physician, examination technician, or the like) as the user. In ultrasound diagnosis of the breast, a transmission/reception surface (more specifically, an acoustic lens surface) of the probe 10 is brought into contact with a surface of the chest of the subject. The examiner scans the probe 10 manually along the surface of the chest while observing a tomographic image which is displayed in real time. When a lesion candidate is identified on the tomographic image, the examiner slowly adjusts a position and posture of the probe 10 and then carefully observes the tomographic image while keeping the position and posture of the probe 10.

In the illustrated configuration example, the probe 10 has a transducer array composed of a plurality of transducers arranged in one dimension. An ultrasound beam (a transmission beam and reception beam) 12 is formed by the transducer array, and a scanning plane 14 is formed by electronic scanning of the ultrasound beam 12. The scanning plane 14 is an observation plane and is a two-dimensional data capture region. As methods of electronic scanning of the ultrasound beam 12, an electronic sector scanning method, an electronic linear scanning method, and the like are known. Convex scanning of the ultrasound beams 12 may be performed. It is also possible to provide a 2D transducer array in the ultrasonic probe 10 and scan an ultrasound beam in two dimensions, thereby obtaining volume data from the inside of the living body.

A positioning system for obtaining position information of the probe 10 may be provided. The positioning system is composed of, for example, a magnetic sensor and a magnetic field generator. The magnetic sensor is attached to the probe 10 (more precisely, a probe head of the probe 10). The magnetic sensor detects a magnetic field generated by the magnetic field generator. In doing so, three-dimensional coordinate information of the magnetic sensor is obtained. A position and posture of the probe 10 are identified based on the three-dimensional coordinate information. Motion information of the probe 10 may be obtained based on information output from the positioning system, and the motion information may be used in mark display control described below.

A transmission circuit 22 functions as a transmission beam former. Specifically, during transmission, the transmission circuit 22 supplies a plurality of transmission signals to the transducer array in parallel. In this manner, a transmission beam is formed. During reception, when waves reflected from the inside of the living body reach the transducer array, a plurality of received signals are output from the plurality of transducers in parallel. A reception circuit 24 functions as a reception beam former and generates beam data by performing phase-alignment and summing (also referred to as delay and summing) of the plurality of received signals.

For each electronic scanning operation, a plurality of sets of beam data arranged in an electronic scanning direction are generated, which constitute received frame data corresponding to the scanning surface 14. Each set of beam data is composed of a plurality of echo data arranged in the depth direction. Although a beam data processing unit is provided downstream of the reception circuit 24, it is omitted from the drawing.

An image forming unit 26 is an electronic circuit that generates a tomographic image (B-mode tomographic image) based on the received frame data. It has a digital scan converter (DSC). The DSC has a coordinate conversion function, a pixel interpolation function, a frame rate conversion function, and the like. More specifically, the image forming unit 26 generates an array of display frame data based on an array of the received frame data. Each set of display frame data constituting the display frame data array is tomographic image data. A plurality of sets of tomographic image data constitute a real-time moving image. Ultrasonic images other than tomographic images may be formed. For example, a color flow mapping image may be formed, or a three-dimensional image expressing a tissue in three dimensions may be formed. In the illustrated configuration example, the display frame data array is sent to a display processing unit 32 and an image analysis unit 28.

The image analysis unit 28 is a module that executes the CADe function. The image analysis unit 28 performs processing of detecting a lesion candidate for each set of frame data; that is, for each tomographic image. Specifically, by performing binarization processing, edge detection processing, or the like on the tomographic image, a lesion candidate, which is a closed area of low brightness, is detected. When the lesion candidate is detected, the image analysis unit 28 outputs lesion candidate information.

The lesion candidate information includes position information and size information of the lesion candidate. The lesion candidate information further includes the reliability. The reliability is a numeric value indicating the possibility that the lesion candidate is an actual lesion. In FIG. 1, a reliability calculation unit 29 that calculates the reliability is shown. The lesion candidate information may include attributes of the lesion candidate (e.g., a disease name, disease state, and the grade of malignancy).

The position information of the lesion candidate is, for example, information indicating the coordinates of a center point of the lesion candidate itself, or information indicating the coordinates of a center point of a figure that is in contact with and surrounds the lesion candidate. The center point is a representative point. The geometric center point of the figure or the center-of-gravity point of the figure may be adopted as the center point. The size information of the lesion candidate is, for example, information indicating the size of the lesion candidate itself, or information indicating the size of a figure that is in contact with and surrounds the lesion candidate. For example, the size of the lesion candidate is identified from the coordinates of the center point of the figure and the coordinates of the upper left corner point of the figure. Assuming that the coordinates of the center point are identified, the coordinates of the upper left corner point may be regarded as the size information of the lesion candidate. The area of the lesion candidate may be obtained as the size information of the lesion candidate. A plurality of lesion candidates may be detected in parallel.

A mark display control unit 30 displays a mark reporting the detected lesion candidate by superimposing the mark on an ultrasonic image. The mark display control unit 30 recognizes that the lesion candidate is detected when the reliability is greater than a predetermined threshold, and generates the mark that surrounds the lesion candidate. The mark display control unit 30 expresses the magnitude of reliability by changing the form of the mark in a stepwise manner. However, a certain period of time (initial stage of detection) from the start of detection is regarded as a fixed form period, and, during the fixed form period, the form of the mark is fixed to the initial form. After the fixed form period elapses, the form of the mark is changed according to the reliability. Changing the form of the mark includes changing the thickness of the line, changing a color of the line, changing the transparency of the line, changing a type of the line, changing the shape of the mark, and the like.

The initial form is less conspicuous; that is, more modest than emphasized forms that may be displayed thereafter. Conversely, the emphasized forms are more recognizable or conspicuous; that is, more noticeable than the initial form.

Some specific examples of form changes will be given. When the reliability is low, the mark may be displayed in a cool color, and when the reliability is high, the mark may be displayed in a warm color. When the reliability is low, the mark may be displayed in low brightness, and when the reliability is high, the mark may be displayed in high brightness. When the reliability is low, the mark may be displayed with high transparency, and when the reliability is high, the mark may be displayed with low transparency. When the reliability is low, the mark may be displayed by a thin line, and when the reliability is high, the mark may be displayed by a thick line. When the reliability is low, the mark may be displayed by a broken line, and when the reliability is high, the mark may be displayed by a solid line. The mark may be switched to another type of mark. For example, display of the four display elements indicating the four corners may be switched to display of a rectangular figure. Several techniques may be applied at the same time.

In the initial stage of detection, detection of the lesion candidate is unstable, and the reliability is also unstable. By limiting changes in form of the mark in the initial stage of detection, it is possible to improve the responsiveness of mark display and prevent the mark from becoming unnecessarily conspicuous and obtrusive in the initial stage of detection.

The image forming unit 26, the image analysis unit 28, and the mark display control unit 30 may be composed of processors. A single processor may function as the image forming unit 26, the image analysis unit 28, and the mark display control unit 30. A CPU described below may function as the image forming unit 26, the image analysis unit 28, and the mark display control unit 30.

The display processing unit 32 has a graphic image generation function, a color calculation function, an image synthesizing function, and the like. The display processing unit 32 receives outputs from the image forming unit 26 and the mark display control unit 30. The mark surrounding the lesion candidate is one of elements constituting a graphic image. Although, in the embodiment, the mark display control unit 30 generates the mark, a main control unit 38, the display processing unit 32, or the like may generate the mark.

A display 36 is composed of an LCD, an organic EL display device, or the like. The display 36 displays tomographic images, which are a moving image, in real time and displays the mark as a part of the graphic image. The display processing unit 32 is composed of, for example, a processor.

The main control unit 38 controls operation of each of the components shown in FIG. 1. In the embodiment, the main control unit 38 is composed of a CPU that executes a program. The main control unit 38 is connected to an operation panel 40. The operation panel 40 is an input device having a plurality of switches, a plurality of buttons, a track ball, a keyboard, or the like.

The operation panel 40 is used to set or change the mark display conditions. The mark display conditions include the fixed form period, a plurality of thresholds, and the like. For example, the fixed form period may be adaptively defined based on the moving speed of the probe 10, the stability of frame data, variation in reliability, or the like.

Although, in the embodiment, the display frame data array is given to the image analysis unit 28, the received frame data array may be given to the image analysis unit 28 (see reference numeral 42). In that case, separately from the image forming unit 26, another image forming unit that easily and quickly performs image formation is provided.

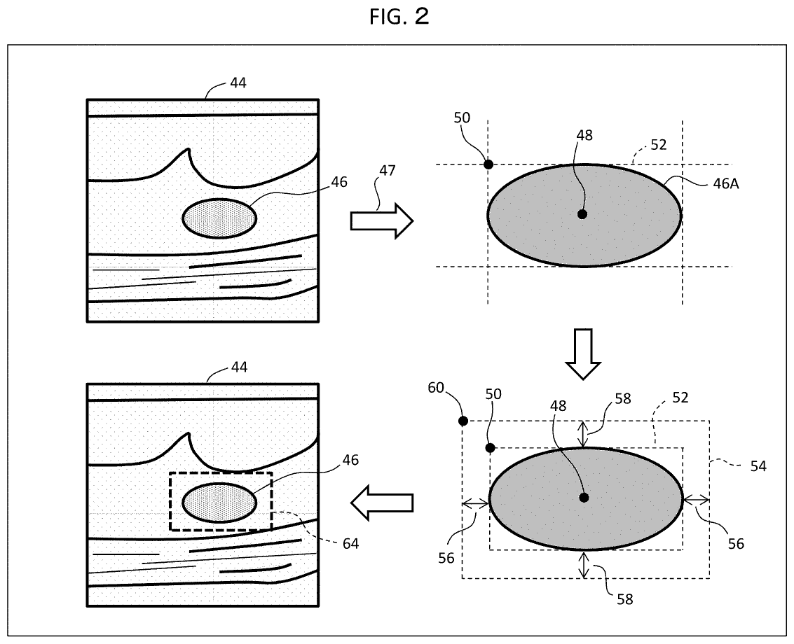

FIG. 2 shows a mark generation method. A tomographic image 44 includes a lesion candidate 46. The tomographic image 44 is subjected to binarization 47, thereby generating a binarized image. The binarized image is subjected to edge detection or area detection, thereby extracting a binarized lesion candidate 46A. A rectangle 52 circumscribing the lesion candidate 46A is defined by, for example, the coordinates of both ends of the lesion candidate 46A in the horizontal direction and the coordinates of both ends of the lesion candidate 46A in the vertical direction. In actual practice, the coordinates of a center point 48 and the coordinates of an upper left corner point 50 of the rectangle 52 are identified.

A rectangle 54 is defined as a figure located outside the rectangle 52 with certain margins 56 and 58 in the horizontal direction and in the vertical direction, respectively, from the rectangle 52. The rectangle 54 is displayed as a mark 64 on the tomographic image 44. The mark 64 is a figure that surrounds the lesion candidate 46 and its periphery. In the illustrated example, the mark 64 is composed of a broken line. A mark composed of only four elements indicating four corner portions may be displayed. A circular or elliptical mark may be displayed.

In the embodiment, detection of the lesion candidate 46 is performed for each set of frame data. When the lesion candidate 46 is detected, the mark 64 is displayed on the tomographic image 44 corresponding to frame data including that lesion candidate 46. By displaying the mark 64, it becomes possible to allow the examiner to perceive the presence of the lesion candidate 46, thereby preventing the lesion candidate 46 from being overlooked.

In a continuous detection state where the lesion candidate 46 is detected continuously, the form of the mark 64 is fixed from the time point when the lesion candidate is started to be detected until the fixed form period elapses. The form of the mark 64 is then changed according to the reliability after the fixed form period elapses. That is, the form of the mark 64 expresses the magnitude of reliability. The mark 64 is erased at the time point when the lesion candidate 46 is no longer detected. In actual practice, the reliability is compared with a first threshold described below, and if the reliability is smaller than the first threshold, it is considered that no lesion candidate is detected. If the reliability is greater than the first threshold, it is considered that a lesion candidate is detected. When detection of the lesion candidate 46 is interrupted for one or several frames, it may be considered that the lesion candidate 46 is detected continuously.

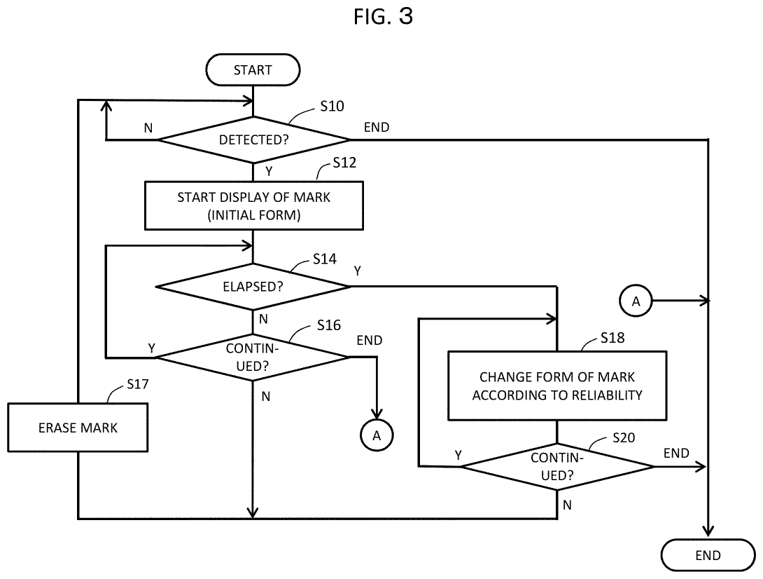

FIG. 3 shows a flowchart of operation of the mark display control unit shown in FIG. 1. In S10, a judgment is made as to whether a lesion candidate is detected. As described above, the presence or absence of a lesion candidate is checked for each set of frame data, and the reliability is referred to for each set of frame data. In the illustrated example, in S10, a judgment of ending of display of the mark may also be obtained.

In S12, display of the mark is started. The initial form is selected as the form of the mark. In S14, a judgment is made as to whether the fixed form period has elapsed from the time point when the lesion candidate was first detected. In S16, a judgment is made as to whether the lesion candidate has been detected continuously until this point. When the lesion candidate has been detected continuously; that is, when a judgment of the continuous detection state is obtained, S14 is repeated. In that process, when the fixed form period has elapsed, S18 is performed. When, in S16, a judgment that detection of the lesion candidate was interrupted is obtained; that is, when the continuous detection state ends, the mark is erased in S17, and then, the steps after S10 are performed. In the illustrated example, in S16, a judgment of ending of display of the mark may also be obtained.

In S18, the form of the mark is changed according to the reliability. In a first example described below, a mark display range defined on the reliability axis is divided into three sections, and three mark forms are assigned to the three sections. In S18, a certain form is selected from the three forms according to the magnitude of reliability, and a mark having that form is displayed. The three forms include the initial form, which corresponds to the lowest section. If, immediately after the fixed form period elapses, the reliability still belongs to the lowest section, the form of the mark is maintained accordingly. This makes it possible to reduce the frequency of changing the form of the mark and achieve both provision of the reliability information and convenience of image observation.

In S20, like in S16 above, a judgment is made as to whether the lesion candidate has been detected continuously. When the lesion candidate has been detected continuously; that is, when a judgment of the continuous detection state is obtained, S18 is repeated. When, in S20, a judgment that detection of the lesion candidate was interrupted is obtained; that is, when the continuous detection state ends, the mark is erased in S17, and the steps after S10 are then performed. In the illustrated example, in S20, a judgment of ending of display of the mark may also be obtained.

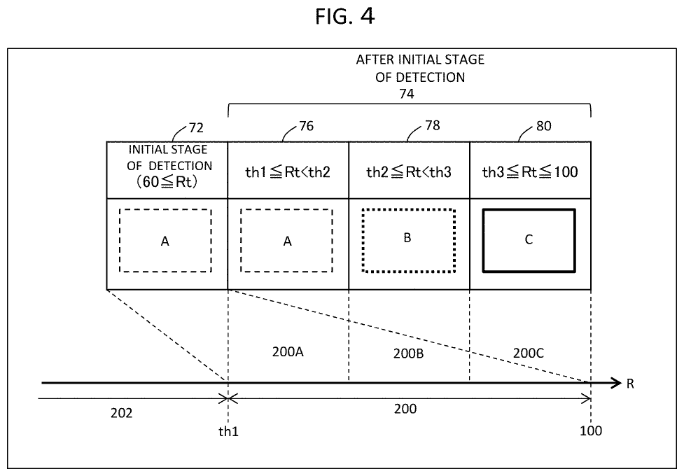

FIG. 4 shows a first example of mark display control. At the bottom of FIG. 4, the reliability axis indicating the magnitude of reliability R is shown. A mark display range 200 and a mark non-display range 202 below the mark display range 200 are defined on the reliability axis. The lower limit of the mark display range 200 is defined by a first threshold th1. The mark display range 200 is divided into a low section 200A, a middle section 200B, and a high section 200C. The low section 200A corresponds to the lowest section. The reliability is a value between 0 and 100. The reliability at a certain timing t is expressed as Rt.

At the initial stage of detection (see reference numeral 72), a mark having a form (initial form) A is displayed. The initial stage of detection corresponds to the fixed form period, and within that period, the mark having the form A is displayed, regardless of the magnitude of reliability Rt. After the initial stage of detection; that is, after the fixed form period elapses (see reference numeral 74), from among the forms A, B, and C, a form corresponding to a section to which the reliability Rt belongs is selected, and a mark having that form is displayed, as shown by reference numerals 76, 78, and 80.

The form A is the initial form described above. When the conspicuousness or the prominence (that is, the degree of noticeability) of the forms is expressed by using inequality signs, the relationship of form A<form B<form C is established. The form A is selected when the reliability Rt is equal to or greater than the first threshold th1 and smaller than a second threshold th2. The form B is selected when the reliability Rt is equal to or greater than the second threshold th2 and smaller than a third threshold th3. The form C is selected when the reliability Rt is equal to or greater than the third threshold th3. For example, the thresholds th1, th2, and th3 are 60, 75, and 90, respectively.

As described above, in the continuous detection state where the lesion candidate is detected continuously, the form of the mark is not changed until a certain period of time elapses after the start of detection of the lesion candidate, even if the reliability changes. The reliability is often unstable during that period, and if the form of the mark is changed according to the reliability, the mark tends to interfere with observation of the ultrasonic image. According to the embodiment, it is possible to avoid such a problem. Further, according to the embodiment, after the certain period of time elapses, it becomes possible to grasp or recognize change in reliability through a change in form of the mark. This makes it possible to closely examine the lesion candidate reported by the mark while taking the reliability into consideration. After the certain period of time elapses, the probe is usually moved slowly or becomes virtually stationary, so that the form of the mark is not changed drastically.

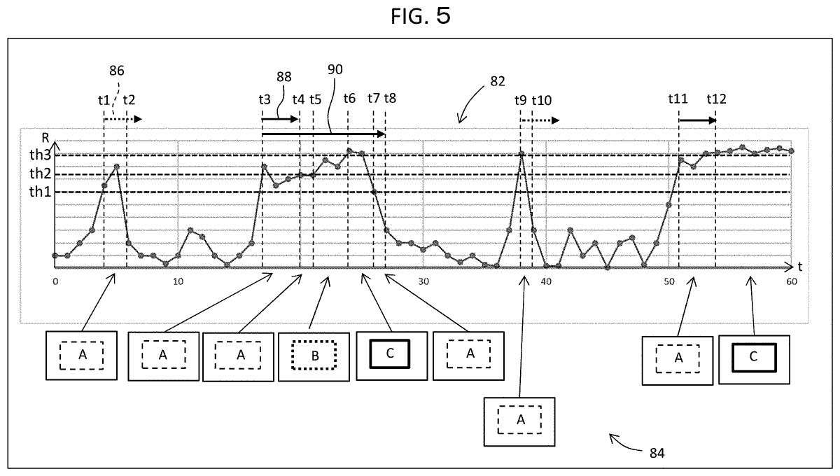

The first example shown in FIG. 4 will be explained in more detail with reference to FIG. 5. Reference numeral 82 indicates a graph showing changes in reliability over time. The horizontal axis is the time axis, and the vertical axis is the reliability axis. The first threshold th1, the second threshold th2, and the third threshold th3 are set on the reliability axis, thereby setting the three sections. Reference numeral 84 indicates marks displayed at certain times.

At time t1, the reliability Rt exceeds the first threshold th1, and a mark having the initial form A is thus displayed. At time t2, the reliability Rt is below the first threshold th1. The period from time t1 to time t2 does not reach a fixed form period 86.

At time t3, the reliability Rt exceeds the first threshold th1 again and also exceeds the second threshold th2. In that case, the mark having the initial form A is displayed. At time t4, a fixed form period 88 has elapsed, and the form of the mark is allowed to be changed from time t4 onward. The fixed form period 88 may be defined to last for several seconds.

At time t4, the reliability Rt belongs to the first section, and the initial form A is maintained. Then, at time t5, the reliability Rt exceeds the second threshold, and, at the time t5, the mark is changed to a mark having the form B. Further, at time t6, the reliability Rt exceeds the third threshold, and the mark is changed to a mark having the form C. Then, at time t7, the reliability Rt returns to the first section, and at that point of time, the mark is changed to the mark having the initial form A. At time t8, the reliability Rt is below the first threshold, and the mark is erased at that point of time.

Then, at time t9, the reliability Rt exceeds the first threshold th1, and actually reaches the third threshold th3. However, at that point of time, the mark having the initial form A is displayed. At time t10, the reliability Rt is below the first threshold th1. This means that the mark is displayed momentarily or only for a short period of time. At time t11, the reliability Rt exceeds the first threshold again. At time t12, the fixed form period has elapsed, and the form of the mark is allowed to be changed.

The fixed form period may be specified by the user or set adaptively and automatically according to the situation. For example, the fixed form period may be defined based on the stability of frame data, the rate of change in reliability distribution throughout the frames, or the like.

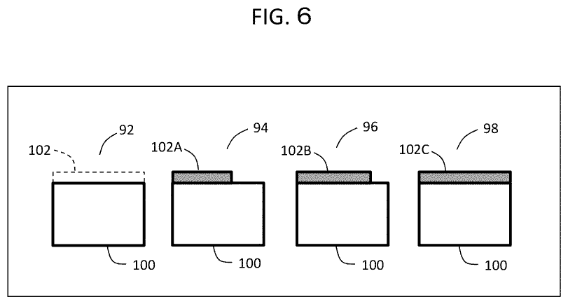

FIG. 6 shows a second example of mark display control. Of forms 92 to 98 that the mark can take, the form 92 is the initial form, and the other three forms 94, 96, and 98 are forms expressing the reliability. When the reliability belongs to the first section after the fixed form period elapses, the form 94 is selected. When the reliability belongs to the second section, the form 96 is selected. When the reliability belongs to the third section, the form 98 is selected. The initial form 92 includes a rectangular mark body 100. An area 102 includes no bar graph.

The forms 94, 96, and 98 have the rectangular mark body 100 and also respectively have bar graphs 102A, 102B, and 102C. The bar length of each of the bar graphs 102A, 102B, and 102C (size in the horizontal direction) expresses the magnitude of reliability. According to the second example, it is possible to intuitively recognize the reliability by observing the form of the mark displayed after the fixed form period elapses. When the bar graphs 102A, 102B, and 102C are displayed, they may be displayed in a semi-transparent manner so that they do not completely hide the ultrasonic image.

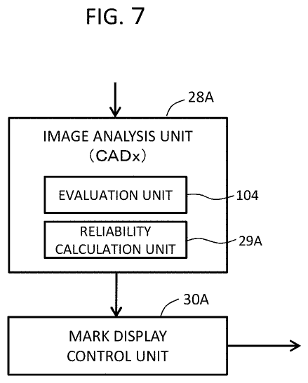

FIG. 7 shows a variation of a part of the configuration shown in FIG. 1. An image analysis unit 28A has an evaluation unit 104 as well as a reliability calculation unit 29A. In the illustrated example, the evaluation unit 104 has a function of distinguishing three classes that are attributes of tumors. Specifically, it has a function of distinguishing among a benign tumor 1 corresponding to a cyst, a benign tumor 2 that is not the benign tumor 1, and a malignant tumor. Among them, for example, the benign tumor 1 is regarded as an unimportant attribute, and the benign tumor 2 and the malignant tumor are regarded as important attributes.

In a candidate detection state, a mark display control unit 30A selects the initial form as the form of the mark until the fixed form period elapses after the start of detection. After that, the mark display control unit 30A hides the mark for the benign tumor 1 and changes the form of the mark for the benign tumor 2 and the malignant tumor according to the reliability. That is, for the lesion candidate having the unimportant attribute, the mark is displayed for a short time to notify the examiner of the presence of the lesion candidate, and then erased from the viewpoint of simplifying the display content. For the lesion candidates having the important attribute, mark display control is performed according to the procedure shown in FIG. 3 and the like.

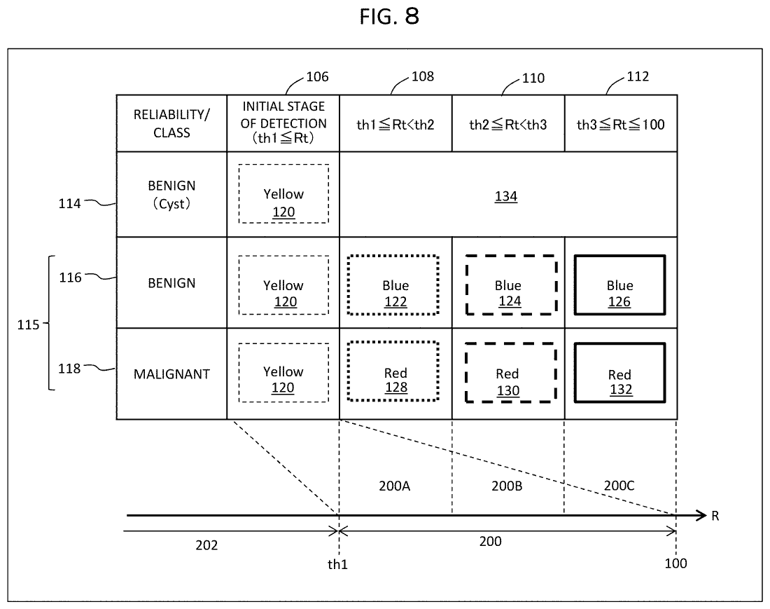

FIG. 8 shows a third example of mark display control. This is based on the configuration shown in FIG. 7. In FIG. 8, the elements that are illustrated in FIG. 4 are assigned the same reference numerals as in FIG. 4, and their description will be omitted.

In FIG. 8, in an initial stage of detection (fixed form period) 106, an initial form 120 is selected as the form of the mark, regardless of an attribute (class). Although, after the initial stage of detection, the form of the mark is changed for a benign tumor 116 and a malignant tumor 118, the mark is erased for a benign tumor 114 (see reference numeral 134). Reference numeral 115 indicates an important attribute corresponding to the tumor 116 and the malignant tumor 118. The benign tumor 114 corresponds to an unimportant attribute. For the important attribute 115, the form of the mark is changed according to the reliability Rt after the initial stage of detection elapses.

Specifically, in the illustrated example, for the benign tumor 116, the mark having a blue color is displayed, and its form (line type) is changed as shown by reference numerals 122, 124, and 126 according to the reliability Rt. For the malignant tumor 118, the mark having a red color is displayed, and its form (line type) is changed as shown by reference numerals 128, 130, and 132 according to the reliability Rt. The form of the mark is selected so as to become more noticeable in a stepwise manner as the reliability Rt increases. In the third example shown in FIG. 8, the forms 122 and 128 corresponding to the lowest section are not the same as the initial form 120. On the other hand, the initial forms 120 are the same as each other, regardless of the differences in attributes.

In the embodiment, when a plurality of lesion candidates are included in the ultrasonic image, the above mark display control is performed for each lesion candidate. The image analysis unit may be composed of a machine learning analyzer. It is, for example, composed of a convolutional neural network (CNN).

1. An ultrasonic diagnostic apparatus comprising:
a calculator that receives an array of frame data obtained by repeating scanning of an ultrasound beam and calculates, for each set of frame data, the reliability indicating the possibility that a lesion candidate included in the frame data is a lesion; and
a controller that displays a mark reporting the lesion candidate on an ultrasonic image formed based on the array of frame data, the controller continuing to display the mark in a continuous detection state where the lesion candidate is detected continuously, wherein
in the continuous detection state, the controller fixes the form of the mark from the time point when the lesion candidate is first detected until a fixed form period elapses, and changes the form of the mark according to the reliability after the fixed form period elapses.

2. The ultrasonic diagnostic apparatus according to claim 1, wherein, in the continuous detection state, the controller changes the form of the mark in a stepwise manner as the reliability increases after the fixed form period elapses.

3. The ultrasonic diagnostic apparatus according to claim 2, wherein
a non-display range and a display range are defined on the reliability axis,
the display range is divided into a plurality of sections including a lowest section,
during the fixed form period, the form of the mark takes an initial form,
after the fixed form period elapses, the form of the mark takes any one of a plurality of forms corresponding to the plurality of sections, and
the plurality of forms include the initial form that corresponds to the lowest section.

4. The ultrasonic diagnostic apparatus according to claim 2, wherein changing the form of the mark in a stepwise manner includes at least one of:
changing the thickness of the mark in a stepwise manner;
changing a color of the mark in a stepwise manner;
changing the transparency of the mark in a stepwise manner; and
changing a type of a line constituting the mark in a stepwise manner.

5. The ultrasonic diagnostic apparatus according to claim 1, wherein
the controller displays the mark when the reliability becomes greater than a threshold and erases the mark when the reliability becomes smaller than the threshold, and
the mark on the ultrasonic image is erased when the reliability becomes smaller than the threshold before the fixed form period elapses.

6. The ultrasonic diagnostic apparatus according to claim 1, wherein
the calculator judges, for each set of frame data, an attribute of the lesion candidate included in the frame data, and
in the continuous detection state, the controller changes the form of the mark according to a combination of the reliability and the attribute after the fixed form period elapses.

7. The ultrasonic diagnostic apparatus according to claim 1, wherein
the calculator judges, for each set of frame data, whether an attribute of the lesion candidate included in the frame data is an important attribute or an unimportant attribute,
when a judgment that the attribute is the important attribute is obtained, the controller changes, in the continuous detection state, the form of the mark according to the reliability after the fixed form period elapses, and
when a judgment that the attribute is the unimportant attribute is obtained, the controller erases, in the continuous detection state, the mark after the fixed form period elapses.

8. A diagnosis assisting method comprising the steps of:
calculating the reliability indicating the possibility that a lesion candidate included in frame data is a lesion, for each set of frame data constituting an array of frame data obtained by repeating scanning of an ultrasound beam; and
displaying a mark reporting the lesion candidate on an ultrasonic image formed based on the array of frame data, wherein
in a continuous detection state where the lesion candidate is detected continuously, the mark is continued to be displayed, and
while the mark is continued to be displayed, the form of the mark is fixed from the time point when the lesion candidate is first detected until a fixed form period elapses and is then changed according to the reliability.

9. A non-transitory storage medium storing a program to be executed in an information processing device, the program comprising the functions of:
calculating the reliability indicating the possibility that a lesion candidate included in frame data is a lesion, for each set of frame data constituting an array of frame data obtained by repeating scanning of an ultrasound beam; and
displaying a mark reporting the lesion candidate on an ultrasonic image formed based on the array of frame data, wherein
in a continuous detection state where the lesion candidate is detected continuously, the mark is continued to be displayed, and
while the mark is continued to be displayed, the form of the mark is fixed from the time point when the lesion candidate is first detected until a fixed form period elapses and is then changed according to the reliability.
